(19) United States
(12) Patent Application Publication
(10) Pub. No.: US 2010/0234892 A1
(43) Pub. Date: Sep. 16, 2010

(54) SPINAL INTERCONNECTING DEVICE AND A STABILIZING SYSTEM USING SAID DEVICE

(76) Inventors: Keyvan Mazda, Paris (FR); Jean-Luc Jouve, Marseille (FR); Richard Minfelde, Paris (FR)

Correspondence Address: SPRINKLE IP LAW GROUP, 1301 W. 25TH STREET, SUITE 408, AUSTIN, TX, 78705, US

(21) Appl. No.: 12/580,202

(22) Filed: Oct. 15, 2009

(30) Foreign Application Priority Data
Oct. 15, 2008 (EP) 08305684

(51) Int. Cl. A61B 17/70

(52) U.S. Cl. 606/276

(57) ABSTRACT

Embodiments disclosed herein provide a spinal interconnecting device and a stabilizing system using the same. The spinal interconnecting device has a connecting body and a hook member fixed to the connecting body for securing the connecting body to a vertebra. The connecting body has a first connecting member and a second connecting member. The first connecting member is adapted to receive the extremity of a spinal construction. The second connecting member is adapted to receive a first end of a separate rod, so that the direction of the separate rod is adaptable with respect to the direction of the spinal construction.

### CROSS-REFERENCE TO RELATED APPLICATION(S)

This application claims a benefit of priority under 35 U.S.C. 119(a) of the filing date of European Patent Application No. 08305684, filed Oct. 15, 2008, entitled “A SPINAL INTERCONNECTING DEVICE AND A STABILIZING SYSTEM USING SAID DEVICE,” which is fully incorporated herein by reference.

### TECHNICAL FIELD OF THE DISCLOSURE

The present invention relates to a device for interconnecting the extremity of a spinal construction assembly with a separate rod and fixing them to a vertebra, and a stabilizing system including said interconnecting device.

One field of application for the invention is holding bones in a relative position, for example, to aid in the healing of breaks or the positioning of bones, or the treatment of scoliosis, or otherwise to correct abnormal curvatures of the spine.

### SUMMARY OF THE DISCLOSURE

The spine is formed of superposed vertebrae, normally aligned along a vertebral axis from the lumbar vertebrae to the cervical vertebrae, each having a posterior wall from which projects a spinous process and two lateral edges from the walls of which project ribs and/or transverse process. Each vertebra also has two lateral pedicles and lamina surfaces.

Accompanying FIG. 1 shows vertebrae V and V′ with the different parts thereof. Reference TP designates the transversal processes, reference P designates the pedicles of the vertebra and reference L designates the lamina.

In order to straighten or stabilize the vertebrae of the spine it is well known to use a stabilizing system which includes an interconnecting longitudinal rod and several fixing elements. Each fixing element is secured to one of the vertebrae to be stabilized and the longitudinal rod is secured to each fixing element. The fixing elements Include a head to be secured to the rod and a fixing member which may consist of a screw (for example a pedicle screw) or a hook. When the patient who is equipped with such a stabilizing system moves his spine, forces are developed by the fixing member of the fixing elements in the vertebrae. In particular, the fixing elements disposed at the extremities of the rod of the stabilizing system or spinal construction assembly develop the greatest forces. It is therefore apparent that it would be advantageous to try to decrease the forces developed at the extremities of the spinal construction assembly.

Moreover, autostable claws are also well known. Such a system is described, for example, in EP 0 571 619. This system includes two fixing elements, each one being provided with a hook member, and a rod to interconnect the fixing elements and, consequently, the vertebrae on which the fixing elements are secured. When defining with the rod the appropriate distance between the two fixing elements, an appropriate stabilization of the vertebrae is obtained.

In another use of the autostable claw system, the hook members of the autostable claw system can be secured to different parts of the same vertebra.

A first object of the present Invention is to provide a device for interconnecting the extremity of a spinal construction assembly with a separate rod, said separate rod being preferably, but not necessarily, part of an autostable claw system, which is more easily implantable by the surgeon than the already known interconnecting devices.

To achieve this goal, according to the invention, the device for interconnecting the extremity of a spinal construction with a separate rod and fixing them to a vertebra comprises a connecting body and a hook member for securing said connecting body to said vertebra, said hook member being fixed to said connecting body, said connecting body comprising a first connecting member, and a second connecting member, said first connecting member being adapted to receive said extremity of the spinal construction, said second connecting member being adapted to receive a first end of said separate rod so that the direction of said separate rod Is adaptable with respect to the direction of said spinal construction.

It is understood that the extremity of the spinal construction system is connected to one end of the separate rod and fixed to the vertebra thanks to a unique device.

Moreover, due to the fact that the second connecting member allows the separate rod to have an adaptable direction with respect to the direction of the spinal construction, the surgeon can choose freely the part of the vertebra to which the interconnecting device is fixed to the vertebra.

Preferably, the second connecting member includes a first rotulating assembly for cooperating with a second rotulating assembly provided at said first end of said separate rod.

According to a preferred embodiment of the invention, said hook member and said connecting body have a common median plane, said first connecting member having an axis and overlapping said hook member, said second connecting member having an axis contained in said median plane and substantially parallel to the axis of the first connecting member and being offset with respect to said first connecting member in a direction perpendicular to the axis of said first connecting member.

It is apparent that, because the second connecting member is offset with respect to the first connecting member in a direction which is perpendicular to the direction of the axis of the connecting member, the thickness of the head of the interconnecting device is not increased.

The hook member can overlap the first connecting member or the second connecting member in a direction of the axes of said connecting member.

A second object of the invention is to provide a stabilizing system which uses an interconnecting device of the type described above.

According to the invention, the stabilizing system comprises a spinal construction assembly having a first end, a separate rod having first and second ends, an interconnecting device of the type defined above for Interconnecting the first end of said spinal construction assembly and the first end of said separate rod, and a fixing element for fixing the second end of said separate rod to a vertebra.

It can be understood that the stabilizing system is basically a combination of a spinal construction system and of an autostable claw system which is formed by the second connecting member of the interconnecting device, the separate rod, and the fixing element. The purpose of the autostable claw system is to permit a decrease in the forces developed within the vertebra by the extremity of the spinal construction system.

Moreover, as the separate rod can have an adaptable direction with respect to the direction of the rod of the spinal construction, the surgeon can freely choose the part of the vertebra to which the interconnecting device is fixed and the part of the vertebra to which the fixing element is secured. This part can be the transverse process, the pedicle or the lamina. The hook member of the interconnecting device and the fixing member of the fixing element (preferably by a hook) can be fixed to two different parts of the same vertebra or to two adjacent different vertebrae.

In both cases, the rods of the spinal construction and of the separate rod can form an angle of less than 90 degrees. This means that the separate rod “returns” towards the rod of the spinal construction.

In both cases, the angle between the two rods can also be more than 90 degrees. This means that the separate rod extends “beyond” the rod of the spinal construction.

These and other aspects and advantages of the invention will be better appreciated and understood when considered In conjunction with the following description and the accompanying drawings. The following description, while indicating various embodiments of the invention and numerous specific details thereof, is given by way of illustration, and not limitation.

### BRIEF DESCRIPTION OF THE DRAWINGS

FIG. 1, already described, is a perspective view of a vertebra showing the different parts of the vertebra.

FIG. 2 is a perspective view of a preferred embodiment of the interconnecting device according to the invention.

FIG. 3 is a sectional view of the connecting device according to plane P of FIG. 2.

FIG. 4 is an exploded view which shows the combination of a spinal construction assembly with a separate rod, the connecting device being provided with a pellicle hook.

FIG. 5 is similar to FIG. 4 but the connecting device is provided with a lamina hook.

FIG. 6 is similar to FIG. 4 but with the components assembled; and

FIGS. 7A to 7D are schematic drawings which show four different combinations of a spinal construction assembly with an autostable claw system.

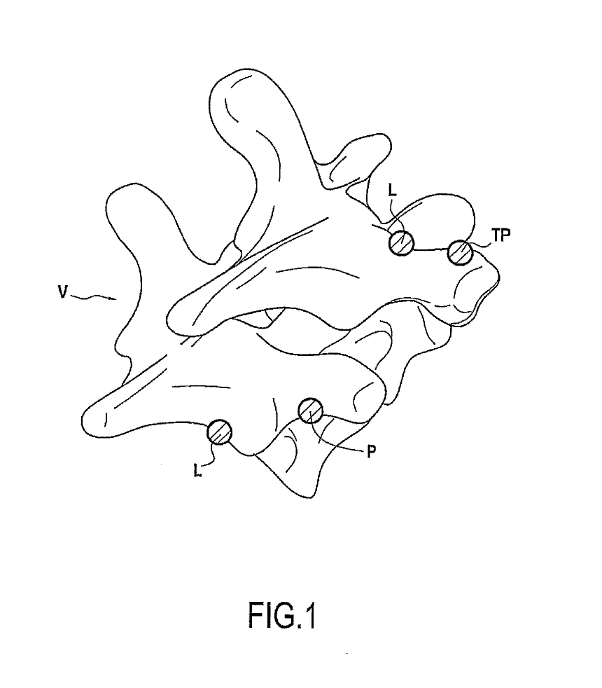

### DETAILED DESCRIPTION

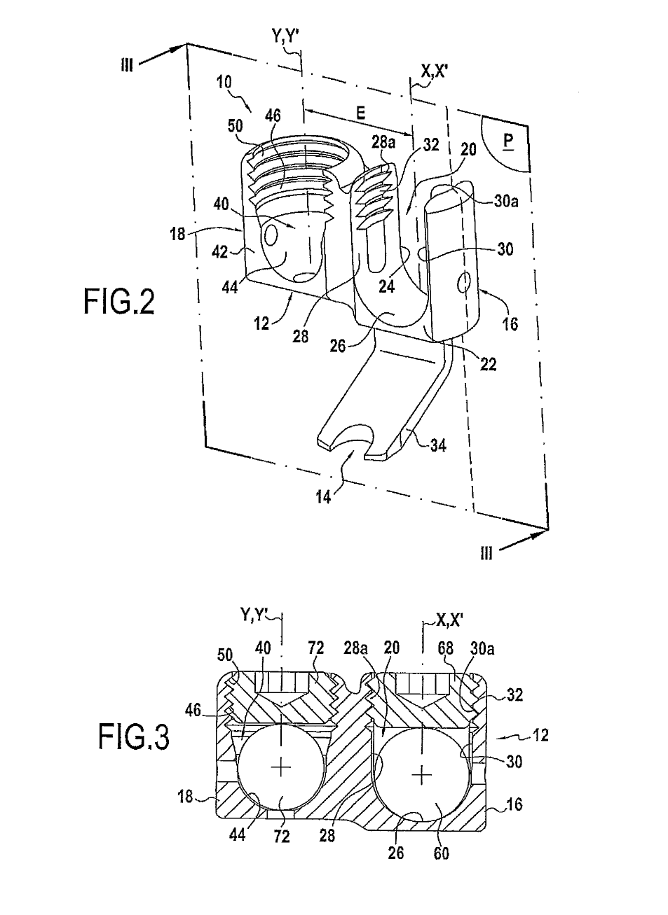

With reference to FIGS. 2 and 3, a preferred embodiment of the interconnecting device will be described.

The interconnecting device 10 comprises a connecting body 12 and a hook member 14.

The connecting body 12 includes a first connecting member 16 and a second connecting member 18. Preferably, the first connecting member 16, the second connecting member 18 and the hook member 14 are formed as a single piece.

Preferably, the first connecting member, which forms a connecting head, is provided with an opening or slot 20 which terminates into two opposable faces 22 and 24 of the head 16. The slot 20 has a bottom wall 26 which is substantially semi-cylindrical and two lateral walls 28 and 30 which are substantially flat.

The upper portion 28a and 30a of the lateral walls 28 and 30 defines portions of cylindrical surfaces. These upper portions 28a and 30a are provided with a threading 32.

Preferably, the hook member 14 is formed by a hook 34. In the case of the embodiment shown by FIG. 2, the hook member 14 is a pedicle hook. However, the hook member 14 might be a lamina hook. The hook 34 is disposed below the first connecting head 14.

Preferably, the second connecting member 18 is provided with an opening 40 which terminates in the face 42 of the second connecting member 18. The bottom 44 of the opening 40 has a wall having substantially the shape of a portion of a half spherical surface. As will be explained hereinafter, the surface 44 forms the female part of a rotulating system.

The upper part 46 of the opening 40 is formed by a wall 48 having the shape of a portion of a cylindrical surface. The upper part 46 is provided with an internal threading 50.

The first connecting member 16 has a longitudinal axis X, X′ and the second connecting member 18 has a longitudinal axis Y, Y′. These two axes are parallel one with the other and are disposed on a plane P. The plane P forms a median plane for the interconnecting device 10. The first and second connecting members 16 and 18 are placed side by side in the direction perpendicular to the direction of the axis X, X′ and Y, Y′. As a result, the axis X, X′ and Y, Y′ are offset by a length E in the plane P.

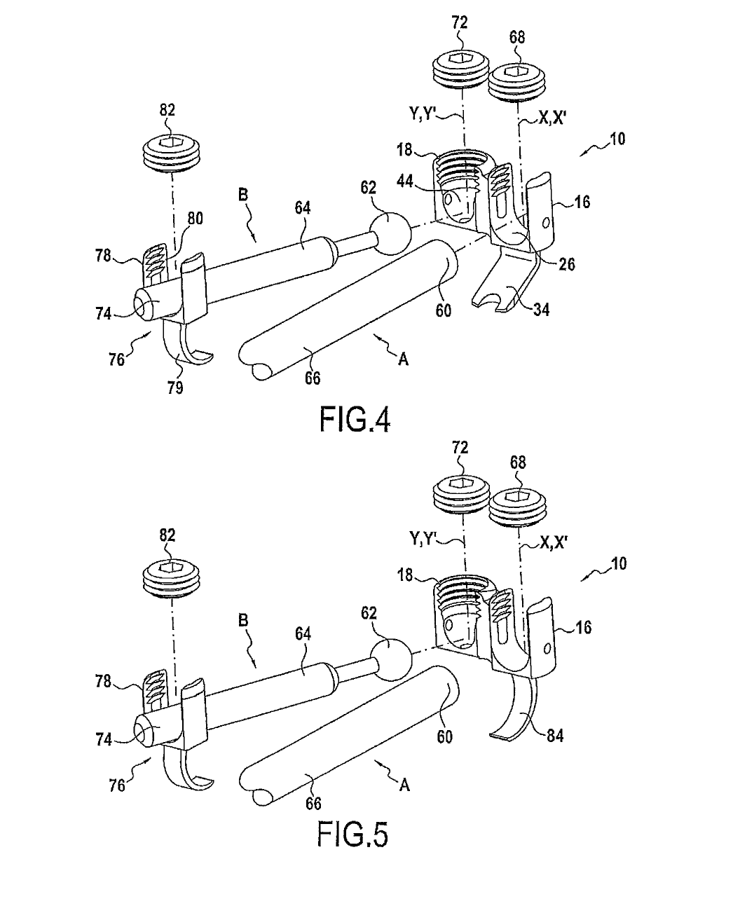

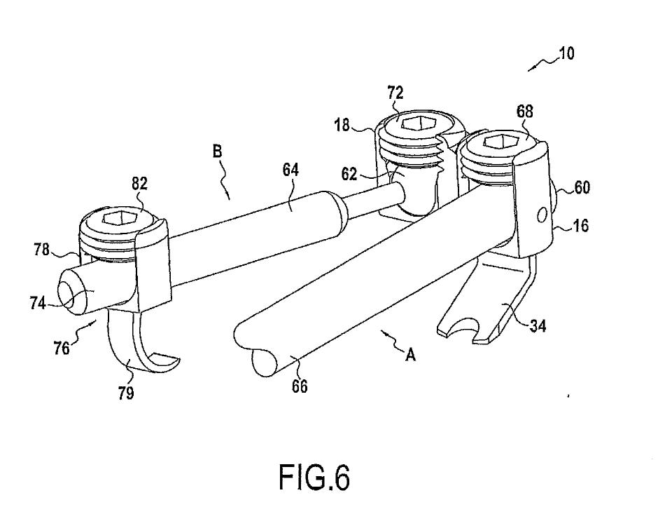

Referring now to FIG. 4, according to a preferred use of an interconnecting device 10 according to the invention, it is used to form a spinal stabilizing system and combines a spinal construction system A, and an adjustable claw system B. The function of the device 10 is to interconnect the extremity 60 of the spinal construction system A with an end 62 of a separate rod 64 which forms a part of the autostable claw B which will be described in more detail hereinafter.

In FIG. 4 the spinal construction A is symbolically represented by a longitudinal connecting rod 66 having the free end 60. As is well known, the entire spinal construction A comprises the connecting rod 66 and a plurality of fixing devices (not shown in the figure) to secure intermediate portions of the rod with the vertebrae to be stabilized by the spinal construction A.

The free end 60 of the rod 66 of the spinal construction A is adapted to be engaged into the opening 20 of the first connecting member 16 of the interconnecting device 10. For this purpose, the diameter of the bottom wail 26 of the opening 20 is slightly greater than the diameter D of the rod 66. When the end 60 of the rod 66 is engaged into the slot 20, this end is connected to the connecting member by means of a screw 68 which cooperates with the threading 32.

As shown in FIG. 4, the end 62 of the separate rod 64 is provided with a spherical member 70 which forms the male part of a rotulating system adapted to be engaged within the partially spherical part 48 of the opening 40 of the second connecting member 1B. The spherical member 70 can be connected to the connecting member 18 by means of a screw 72 which can cooperate with the threading 50 of the opening of connecting member 18.

The second end 74 of the separate rod 64 can be connected to a fixing element 76. Preferably, the fixing element 76 comprises a connecting head 78 provided with a slot 80 to receive the rod 64 and a hook 79 to fix the fixing element 76 to a vertebra. In the example shown in FIG. 4, the hook 79 is a lamina hook. The end 74 of the rod 64 can be secured to the head 78 by means of a screw 82. In FIG. 5, the connecting device is provided with a lamina hook 84.

In view of the above description, it is clear that a preferred use of the interconnecting device 10 is to interconnect an autostable claw system B and a spinal construction system A.

The autostable claw system consists in the rod 64, the fixing element 76 with its hook 79, and the second connecting member 18 and the hook 34 of the interconnecting device 10.

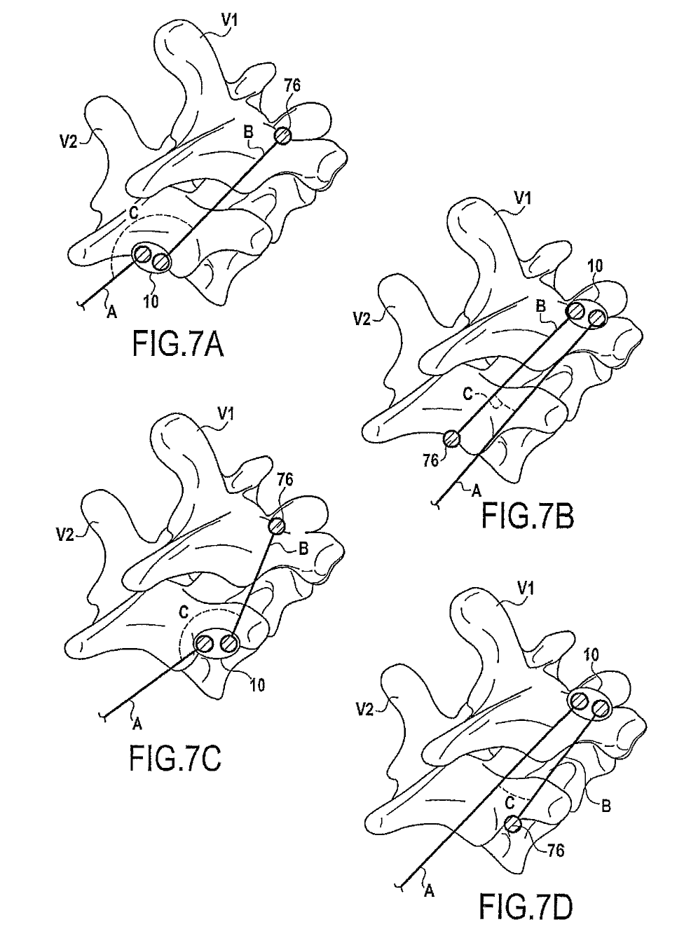

As already explained, the connection between the end 62, 70 of the separate rod 64 and the second connecting member 18 is achieved by a rotulating system consisting in the spherical member 70 and the partially spherical bottom wall 44 of the connecting member 18. As a result, the direction of the separate rod 64 can be chosen by the surgeon before screwing the screw 72 with the connecting member 18. As a result, the surgeon can freely choose the part of the vertebra to which the fixing element 76 is fixed. This feature significantly simplifies the action of the surgeon as will be explained below with reference to FIGS. 7A and 7D.

Moreover, the two connecting members 16 and 18 are disposed side by side. Consequently, the interconnecting device is less aggressive than a device where the two connecting members are superposed.

FIGS. 7A to 7D illustrate the great number of possible positions of the autostabie claw system B and the spinal construction A. These figures also illustrate the great number of possible fixing locations of the stabilizing system to the vertebrae.

In the case of FIG. 7A, the interconnecting device 10 and the fixing element 76 are both fixed to a lamina of vertebrae V1 and V2, and the autostable claw system B extends beyond the spinal construction A. The angle C between the rods of the systems A and B is substantially equal to 180 degrees.

In the case of FIG. 7B, the interconnecting device 10 and the fixing element 76 are still secured to a lamina of vertebrae VI and V2 but the angle C between the autostable claw system B and the spinal construction A is greatly reduced. The rods of the systems A and B are substantially parallel and the system B “returns” towards the system A. In the case of FIG. 7C, the fixing element 76 is fixed to a lamina of vertebra VI and the interconnecting device 10 is fixed to a pedicle of vertebra V2 and the angle C is approximately 150 degrees.

Finally, in the case of FIG. 7D, the interconnecting device 10 is fixed to a lamina of vertebra V1 and the fixing element 76 is secured to a pedicle of vertebra V2. The angle C is about 10 degrees.

In the above description, the fixing element 76 and the Interconnecting device 10 are fixed to two different vertebrae. Obviously, these two securing components can be fixed to two different parts of the same vertebra. In this case, the rod 64 of the autostable claw system B has a reduced length.

However, in both cases, the anchoring of the extremity of the spinal construction A into the vertebra is improved by the provision of the autostable claw system B.

Moreover, in the above description, the hook member 34 or 84 has the same axis (XX′) as the first connecting member 16. Of course, the hook member could be “aligned” with the second connecting member 18.

The advantage of this variant of the invention has exactly the same advantages as the previously described preferred embodiments.

What is claimed is:

1. A device for interconnecting the extremity of a spinal construction with a separate rod and fixing them to a vertebra comprising:
a connecting body; and
a hook member for securing said connecting body to said vertebra, said hook member being fixed to said connecting body;
said connecting body comprising:
a first connecting member; and
a second connecting member, said first connecting member being adapted to receive said extremity of the spinal construction; said second connecting member being adapted to receive a first end of said separate rod, so that the direction of said separate rod is adaptable with respect to the direction of said spinal construction.

2. The device according to claim 1, wherein said second connecting member includes a first rotulating assembly for cooperating with a second rotulating assembly provided at said first end of said separate rod.

3. The device according to claim 1, wherein
said hook member and said connecting body have a common median plane;
said first connecting member having an axis; and
said second connecting member having an axis contained in said median plane and substantially parallel to the axis of the first connecting member and being offset with respect to said first connecting member in a direction perpendicular to the axis of said first connecting member.

4. The device according to claim 1, wherein said second connecting member is adapted to receive the first end of said separate rod on both sides with respect to said median plane.

5. The device according to claim 1, wherein said hook member is a lamina hook member.

6. The device according to claim 1, wherein said hook member is a pedicle hook member.

7. The device according to claim 1, wherein said first connecting member overlaps said hook member according to the direction of said axes.

8. A stabilizing system comprising:
a spinal construction assembly having a first end;
a separate rod having first and second ends;
an interconnecting device for interconnecting the first end of said spinal construction assembly and the first end of said separate rod; and
a fixing element for fixing the second end of said separate rod to a vertebra, wherein said interconnecting device comprises:
a connecting body; and
a hook member for securing said connecting body to said vertebra, said hook member being fixed to said connecting body;
said connecting body comprising:
a first connecting member; and
a second connecting member, said first connecting member being adapted to receive said extremity of the spinal construction; said second connecting member being adapted to receive a first end of said separate rod, so that the direction of said separate rod is adaptable with respect to the direction of said spinal construction.

9. The stabilizing system according to claim 8, wherein said fixing element includes a hook member.

10. The stabilizing system according to claim 9, wherein said hook member of said fixing element is a lamina hook member.

11. The stabilizing system according to claim 9, wherein said hook member of said fixing element is a pedicle hook member.

12. The stabilizing system according to claim 8, wherein said interconnecting device and said fixing element form an autostable claw system.

13. The stabilizing system according to claim 8, wherein the angle between the spinal construction assembly and said separate rod is less than 90 degrees.

14. The stabilizing system according to claim 8, wherein the angle between said spinal construction assembly and said separate rod is more than 90 degrees.

15. The stabilizing system according to claim 8, wherein said separate rod is designed so that said interconnecting device and said fixing element are mounted on the same vertebra.

16. The stabilizing system according to claim 8, wherein said separate rod is designed so that said interconnecting device and said fixing device are mounted on two adjacent vertebrae.
